(19) United States
(12) Patent Application Publication
(10) Pub. No.: US 2021/0008239 A1
(43) Pub. Date: Jan. 14, 2021

(54) Medical Ozone Sterile Isotonic Saline Solution And Preparation Method And Application Thereof

(71) Applicant: Zhiyong YANG, Xining (CN)

(72) Inventor: Zhiyong YANG, Xining (CN)

(21) Appl. No.: 16/531,121

(22) Filed: Aug. 5, 2019

(30) Foreign Application Priority Data
Jul. 8, 2019 (CN) 201910610695.8

(51) Int. Cl. A61L 2/18

(52) U.S. Cl. CPC A61L 2/183

(57) ABSTRACT

A medical ozone sterile isotonic saline solution, a preparation method and an application thereof are provided. The medical ozone sterile isotonic saline solution is composed of medical ozone and a 0.9% sodium chloride injection. A ratio of a mass of the medical ozone to a volume of the 0.9% sodium chloride injection is equal to 3 μg/100 ml. The medical ozone is prepared into a medical injection (sterile isotonic saline solution) under normal temperature and normal pressure according to a specific ratio. The medical injection has stable properties, which is conductive to transportation and presents safety and effectiveness during use. The medical injection can be mass-produced, which is beneficial for clinical use and promotion.

### CROSS REFERENCE TO THE RELATED APPLICATIONS

This application is based upon and claims priority to Chinese Patent Application No. 201910610695.8, filed on Jul. 8, 2019, the entire contents of which are incorporated herein by reference.

### TECHNICAL FIELD

The present disclosure relates to the technical field of medicine, in particular to a medical ozone sterile isotonic saline solution and a preparation method and an application thereof.

### BACKGROUND

Ozone gas is a broad-spectrum, high-efficiency, rapid and safe sterilizing gas, which can kill various pathogens such as bacterial spores, viruses, fungi, etc, and has the functions of improving human blood oxygenation, promoting nerve growth and repair, lowering blood fat, treating diabetes and hepatitis, anti-tumor, treating disc herniation and cerebral stroke, enhancing immunity, promoting wound healing, anti-infection, anti-inflammatory, pain relief, etc.

Sodium chloride injection (saline solution) is a traditional product commonly used in the medical field around the world.

At present, in the technical field of medicine, there is no data and record on isotonic sterile injection drugs prepared by using the ozone and the sodium chloride injection as raw materials and a clinical validation thereof on application effect.

### SUMMARY

The objective of the present disclosure is to provide a medical ozone sterile isotonic saline solution and a preparation method and an application thereof.

To achieve the above-mentioned objective, the technical solution of the present disclosure is as follows.

A medical ozone sterile isotonic saline solution is composed of medical ozone and 0.9% sodium chloride injection.

Preferably, a ratio of a mass of the medical ozone to a volume of the 0.9% sodium chloride injection is equal to 3 μg/100 ml.

The present disclosure also discloses a method of preparing the above-mentioned medical ozone sterile isotonic saline solution, including the following steps:

(1) preparing a medical oxygen supply equipment with a pressure of 13.5 MPa and an oxygen content of 99.5%, two disposable syringes, a bottle of 0.9% sodium chloride injection in a 250 ml plastic bottle, and a medical ozone therapeutic device, connecting the medical oxygen supply equipment to the medical ozone therapeutic device properly;

(2) firstly, using a disposable syringe to extract 150 ml of the 0.9% sodium chloride injection from the 250 ml of 0.9% sodium chloride injection, and leaving 100 ml of the 0.9% sodium chloride injection in the plastic bottle; then, turning on a power and an oxygen switch of the medical ozone therapeutic device, adjusting an ozone concentration to 30 mg/L, and using a disposable syringe to collect 100 ml of the medical ozone and injecting into the plastic bottle containing 100 ml of 0.9% sodium chloride injection;

(3) shaking the plastic bottle injected with 100 ml medical ozone manually or mechanically for 1-3 minutes and using immediately.

Another objective of the present disclosure is to provide an application of the above-mentioned medical ozone sterile isotonic saline solution in a treatment of varicose veins.

A further objective of the present disclosure is to provide an application of the above-mentioned medical ozone sterile isotonic saline solution in a treatment of an acute diarrhea.

Compared with the prior art, the advantages and positive effects of the present disclosure are as follows.

The medical ozone is prepared into a medical injection (sterile isotonic saline solution) under normal temperature and normal pressure according to a specific ratio. The medical injection has stable properties, which is conductive to transportation and presents safety and effectiveness during use. The medical injection can be mass-produced, which is beneficial for clinical use and promotion. Moreover, it is economical, concise and efficient, making it a reality for patients to benefit directly from the ozone sterile isotonic saline solution, and opening up a new approach for the preparation and application of ozone in the medical field.

### DETAILED DESCRIPTION OF THE EMBODIMENTS

Specific embodiments of the present disclosure are described in detail below.

### Embodiment

A medical ozone sterile isotonic saline solution is composed of medical ozone and 0.9% sodium chloride injection; a ratio of a mass of the medical ozone to a volume of the 0.9% sodium chloride injection is equal to 3 μg/100 ml.

A method of preparing the above-mentioned medical ozone sterile isotonic saline solution includes the following steps:

(1) A medical oxygen supply equipment-oxygen cylinder with a pressure of 13.5 MPa and an oxygen content of 99.5% meeting the 2015 National Pharmacopoeia GB8982-2010 was prepared;

Two disposable syringes were prepared;

A bottle of 0.9% sodium chloride injection in a 250 ml plastic bottle with a State Food and Drug Administration (SFDA) approval number was prepared;

A medical ozone therapeutic device meeting the national machinery standard 20183261534 was prepared, which is produced by Zibo Yuehua Medical Instrument Co., Ltd. The product model is CHY-31, and the product performance is as follows: an output concentration of ozone is 5 mg/L-80 mg/L, an ozone output is continuous and adjustable, and an error is less than or equal to 3%;

The medical oxygen supply equipment was connected properly to the medical ozone therapeutic device;

(2) Firstly, a disposable syringe was used to extract 150 ml of 0.9% sodium chloride injection from the 250 ml of 0.9% sodium chloride injection in a 250 ml plastic bottle, and 100 ml of 0.9% sodium chloride injection was left in the plastic bottle; then, a power and an oxygen switch of the medical ozone therapeutic device were turned on, an ozone concentration was adjusted to 30 mg/L, and a disposable syringe was used to collect 100 ml of medical ozone to inject into the plastic bottle containing 100 ml of 0.9% sodium chloride injection;

(3) The plastic bottle injected with 100 ml medical ozone was shaken mechanically for 2 minutes, the medical ozone and the 0.9% sodium chloride injection form a large number of tiny bubbles and a surface of gas-liquid mixture is instantaneously increased, so as to prepare the medical ozone sterile isotonic saline solution for instant use.

### Application Example 1

100 ml of the medical ozone sterile isotonic saline solution prepared according to the method of the embodiment was injected from a distal end of varicose veins of lower extremity, intravenously, once a day, and symptoms of the varicose veins of the lower extremity were improved after 7-10 days.

### Application Example 2

250 ml of the medical ozone sterile isotonic saline solution prepared according to the method of the embodiment was injected from a dorsal vein of an upper limb, intravenously, once a day, and symptoms of acute diarrhea were improved after 3-5 days.

Clinical Summary

Through the summary of clinical examples, the safety and effects of the medical ozone sterile isotonic saline solution “ozone+0.9% sodium chloride injection” of the present disclosure on clinical application can be further described and confirmed. Since the product was used in clinical application for radically curing neck, shoulder, waist and leg disease, vaginitis, cervicitis, eczema and Helicobacter pylori, promoting tissue repair and healing of fractures and pressure sores, and treating conjunctivitis, halitosis, tinea manus and pedis, abscess after surgery, acute cerebral infarction, varicose veins and other diseases in 1999, a total of 9199 patients with various diseases that have been treated with this product formulation have been recorded, wherein, 5011 patients are male, 4188 patients are female, the youngest patient is 12 years old, the oldest patient is 88 years old, 613 patients are under 18 years old, 1518 patients are aged 19-30, 2785 patients are aged 31-49, 3192 patients are aged 50-69, and 1091 patients are over 70 years old. At normal temperature and pressure, the product was intravenously injected, or wetly applied on a surface of a sore skin as an external use, washing the patient's body cavity and abscess, and reaching the tumor site etc. by an intravenous injection.

In the treatment of pain or discomfort of 218 patients caused by cervical spondylosis, significant effects were achieved in 159 patients, general effects were achieved in 59 patients, and 0 patients were ineffective.

In the treatment of pain of 326 patients caused by simple shoulder lesion, significant effects were achieved in 296 patients, general effects were achieved in 30 patients, and 0 patients were ineffective.

In the treatment of pain of 55 patients caused by simple mesh elbow inflammation, significant effects were achieved in 27 patients, general effects were achieved in 28 patients, and 0 patients were ineffective.

In the treatment of headache of 133 patients caused by various diseases, significant effects were achieved in 69 patients, general effects were achieved in 64 patients, and 0 patients were ineffective.

In the treatment of 880 patients with waist disease (disc disease, etc.), significant effects were achieved in 420 patients, general effects were achieved in 460 patients, and 0 patients were ineffective.

In the treatment of 985 patients with knee pain caused by various causes, significant effects were achieved in 529 patients, general effects were achieved in 456 patients, and 0 patients were ineffective.

In the treatment of 528 patients with pain caused by internal diseases and related diseases, significant effects were achieved in 392 patients, general effects were achieved in 136 patients, and 0 patients were ineffective.

In the treatment of 1009 patients with vaginitis, significant effects were achieved in 692 patients, general effects were achieved in 317 patients, and 0 patients were ineffective.

In the treatment of 805 patients with cervicitis, significant effects were achieved in 503 patients, general effects were achieved in 302 patients, and 0 patients were ineffective.

In the treatment of 765 patients with eczema, significant effects were achieved in 304 patients, general effects were 461 patients, and 0 patients were ineffective.

In the treatment of 159 patients with pressure sore, significant effects were achieved in 79 patients, general effects were achieved in 80 patients, and 0 patients were ineffective.

In the treatment of 113 patients after surgery of perianal abscess, significant effects were achieved in 62 patients, general effects were achieved in 51 patients, and 0 patients were ineffective.

In the treatment of 214 patients after surgery of fracture, significant effects were achieved in 109 patients, general effects were achieved in 105 patients, and 0 patients were ineffective.

In the treatment of 1046 patients infected with Helicobacter pylori, significant effects were achieved in 498 patients, general effects were achieved in 548 patients, and 0 patients were ineffective.

In the treatment of 560 patients with conjunctivitis, significant effects were achieved in 313 patients, general effects were achieved in 247 patients, and 0 patients were ineffective.

In the treatment of 867 patients with halitosis, significant effects were achieved in 506 patients, general effects were achieved in 361 patients, and 0 patients were ineffective.

In the treatment of 510 patients with tinea manus and pedis, significant effects were achieved in 297 patients, general effects were achieved in 213 patients, and 0 patients were ineffective.

In the treatment of 244 patients with varicose veins, significant effects were achieved in 165 patients, general effects were achieved in 79 patients, and 0 patients were ineffective.

The above-mentioned embodiments are only preferred embodiments of the present disclosure, and any simple modifications, changes, and substitutions to the above-mentioned embodiments in accordance with the technical essence of the present disclosure are within the scope of the technical solutions of the present disclosure.

What is claimed is:

1. A medical ozone sterile isotonic saline solution, wherein the medical ozone sterile isotonic saline solution is composed of medical ozone and a 0.9% sodium chloride injection.

2. The medical ozone sterile isotonic saline solution of claim 1, wherein a ratio of a mass of the medical ozone to a volume of the 0.9% sodium chloride injection is equal to 3 μg/100 ml.

3. A method of preparing a medical ozone sterile isotonic saline solution, comprising the following steps:
(1) injecting medical ozone into a container containing a 0.9% sodium chloride injection;
(2) shaking the container to dissolve the medical ozone in the 0.9% sodium chloride injection.

4. The method of preparing the medical ozone sterile isotonic saline solution of claim 3, further comprising the following steps:
(1) preparing a medical oxygen supply equipment with a pressure of 13.5 MPa and an oxygen content of 99.5%, two disposable syringes, 250 ml of the 0.9% sodium chloride injection in a 250 ml plastic bottle, and a medical ozone therapeutic device, connecting the medical oxygen supply equipment to the medical ozone therapeutic device properly;
(2) firstly, using one of the two disposable syringes to extract 150 ml of the 0.9% sodium chloride injection from the 250 ml of the 0.9% sodium chloride injection in the 250 ml plastic bottle, and leaving 100 ml of the 0.9% sodium chloride injection in the 250 ml plastic bottle; then, turning on a power and an oxygen switch of the medical ozone therapeutic device, adjusting an ozone concentration to 30 mg/L, and using another one of the two disposable syringes to collect 100 ml of the medical ozone and injecting the 100 ml medical ozone into the 250 ml plastic bottle containing the 100 ml of the 0.9% sodium chloride injection;
(3) shaking the 250 ml plastic bottle injected with the 100 ml medical ozone manually or mechanically for 1-3 minutes to obtain the medical ozone sterile isotonic saline solution, and using the medical ozone sterile isotonic saline solution immediately.

5. A method of treating varicose veins, comprising using the medical ozone sterile isotonic saline solution of claim 1.

6. A method of treating an acute diarrhea, comprising using the medical ozone sterile isotonic saline solution of claim 1.
